SEQUENCE LISTING (10 sequences)

SEQ ID NO: 1 - protein, 73 aa, Homo sapiens
MLGLVLALLSSSSAEEYVGLSANQCAVPAKDRVDCGYPHVTPKECNNRGCCFDSRIPGVPWCFKPLQEAECTF

SEQ ID NO: 2 - DNA, 222 bp, Homo sapiens
atgctggggctggtcctggccttgctgtcctccagctctgctgaggagtacgtgggcctgtctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcagaatgcaccttctga

SEQ ID NO: 3 - protein, 84 aa, Homo sapiens
MATMENKVICALVLVSMLALGTLAEAQTETCTVAPRERQNCGFPGVTPSQCANKGCCFDDTVRGVPWCFYPNTIDVPPEEECEF

SEQ ID NO: 4 - DNA, 255 bp, Homo sapiens
atggccaccatggagaacaaggtgatctgcgccctggtcctggtgtccatgctggccctcggcaccctggccgaggcccagacagagacgtgtacagtggccccccgtgaaagacagaattgtggttttcctggtgtcacgccctcccagtgtgcaaataagggctgctgtttcgacgacaccgttcgtggggtcccctggtgcttctatcctaataccatcgacgtccctccagaagaggagtgtgaattttag

SEQ ID NO: 5 - protein, 106 aa, Homo sapiens [sequence omitted]

SEQ ID NO: 6 - DNA, 390 bp, Homo sapiens
atgggacggcgagacgcccagctcctggcagcgctcctcgtcctggggctatgtgccctggcggggagtgagaaaccctccccctgccagtgctccaggctgagcccccataacaggacgaactgcggcttccctggaatcaccagtgaccagtgttttgacaatggatgctgtttcgactccagtgtcactggggtcccctggtgtttccaccccctcccaaagcaagagtcggatcagtgcgtcatggaggtctcagaccgaagaaactgtggctacccgggcatcagccccgaggaatgcgcctctcggaagtgctgcttctccaacttcatctttgaagtgccctggtgcttcttcccgaagtctgtggaagactgccattactaa

SEQ ID NO: 7 - protein, 41 aa, Artificial Sequence
  based on Homo sapiens
XCTVAPRERQNCGFPGVTPSQCANKGCCFDDTVRGVPWCFX

SEQ ID NO: 8 - protein, 42 aa, Artificial Sequence
  based on Homo sapiens
XCSRLSPHNRTNCGFPGITSDQCFDNGCCFDSSVTGVPWCFX

SEQ ID NO: 9 - protein, 41 aa, Artificial Sequence
  based on Homo sapiens
XCVMEVSDRRNCGYPGISPEECASRKCCFSNFIFEVPWCFX

SEQ ID NO: 10 - protein, 41 aa, Artificial Sequence
  based on Homo sapiens
XCAVPAKDRVDCGYPHVTPKECNNRGCCFDSRIPGVPWCFX

(19) United States
(12) Patent Application Publication
(10) Pub. No.: US 2006/0189526 A1
(43) Pub. Date: Aug. 24, 2006

(54) COMPOSITIONS CONTAINING AN INTESTINAL TREFOIL PEPTIDE AND A MUCOADHESIVE

(76) Inventor: Daniel K. Podolsky, Wellesley, MA (US)

Correspondence Address: CLARK & ELBING LLP, 101 FEDERAL STREET, BOSTON, MA, 02110, US

(21) Appl. No.: 11/275,600

(22) Filed: Jan. 18, 2006

Related U.S. Application Data
(63) Continuation of application No. 10/434,752, filed on May 9, 2003.
(63) Continuation-in-part of application No. 10/131,063, filed on Apr. 24, 2002, now abandoned.
(60) Provisional application No. 60/422,708, filed on Oct. 31, 2002.

(51) Int. Cl. A61K 38/17; A61K 31/715

(52) U.S. Cl. 514/12; 514/54

(57) ABSTRACT

The invention features compositions containing an intestinal trefoil peptide and a mucoadhesive excipient. Such compounds are useful, e.g., for the treatment or prevention of lesions. Compositions containing an intestinal trefoil peptide and a mucoadhesive excipient may be formulated in combination with one or more additional therapeutic agents.

### CROSS REFERENCE TO RELATED APPLICATIONS

This application is a continuation of U.S. application Ser. No. 10/434,752, filed May 9, 2003, which claims the benefit of U.S. Provisional Application No. 60/422,708, filed Oct. 31, 2002. U.S. application Ser. No. 10/434,752 is a continuation-in-part of U.S. application Ser. No. 10/131,063, now abandoned, filed Apr. 24, 2002. The disclosures of each of the aforementioned applications are hereby incorporated by reference.

### FIELD OF THE INVENTION

This invention provides methods and compositions for treating lesions of the upper alimentary canal including the oral cavity and esophagus.

### BACKGROUND OF THE INVENTION

Oral mucositis is the destruction of the oral mucosal epithelium which results in erythema, ulcerations, and pain in the oral cavity. Mucositis often arises as a complication of antineoplastic therapy such as cancer chemotherapy or radiotherapy. The painful ulcerative lesions of mucositis can cause patients to restrict their oral intake of food and liquids; as a result, they lose weight and suffer from dehydration. Severe mucositis can necessitate the de-escalation or the complete interruption of anti-neoplastic therapy. Chemotherapy or radiotherapy can also disrupt mucosal epithelium more distally in the gastrointestinal tract including the esophagus, stomach, and small and large intestines, resulting in pain and organ dysfunction (i.e., diarrhea).

The mucositis lesions are also sites of secondary infections, acting as portals of entry for endogenous oral microorganisms; a particularly serious concern in patients who are immunocompromised. Mucositis is therefore a significant risk factor for chronic debilitating local infections (e.g. yeast (Candida) infections) as well as life-threatening systemic infection (septicemia). Patients with mucositis and neutropenia have a relative risk of septicemia that is at least four times greater than that of individuals without mucositis.

Aphthous ulcers of the mouth (or aphthous stomatitis) are a common and painful problem; approximately 10% of the population suffers from these mouth sores at one time or another. The cause of outbreaks of aphthous sores are not well understood, although they tend to be associated with stress and minor injury to the inside of the mouth. No satisfactory treatments are available, although topical application of steroids provides relief for some patients.

### SUMMARY OF THE INVENTION

This invention features a method for treating a lesion of the upper alimentary canal in a mammal by administering to the mammal a therapeutically effect amount of a trefoil peptide. Preferably, the mammal is a human. Treatment or prevention of lesions according to the invention can speed healing, reduce pain, delay or prevent occurrence of the lesion, and inhibit expansion, secondary infection, or other complications of the lesion. Preferably, the mammal is a human. In particularly useful embodiments, the trefoil peptide is SP, pS2, ITF, ITF₁₅₋₇₃, ITF₂₁₋₇₃, ITF₁₋₇₂, ITF₁₅₋₇₂, or ITF₂₁₋₇₂, and is present in a pharmaceutical composition containing a pharmaceutically acceptable carrier. Other useful trefoil peptides include polypeptides that are substantially identical to SP, pS2, ITF, ITF15-73, ITF₂₁₋₇₃, ITF₁₅₋₇₂, or ITF₂₁₋₇₂. The trefoil peptide may be administered as a monomer, a dimer, or another multimeric form.

Lesions of the upper alimentary canal such as mucositis, aphthous stomatitis, and gingivitis can be treated by the methods of this invention. Additionally, lesions of the upper alimentary canal that result from antineoplastic therapy (i.e., chemotherapy or radiotherapy), Behcet's Disease, biopsy, surgery, tumor resection, thermal or chemical burns, tooth extraction, trauma from any cause, or lesions caused by microbial (i.e., bacterial, viral, or fungal) infection are also amenable to treatment.

In preferred embodiments, the patient is also administered a second therapeutic agent. Preferred second therapeutic agents include anti-inflammatory agents, antibacterial agents (i.e., penicillins, cephalosporins, tetracyclines, or aminoglycosides), antifungal agents (i.e., nystatin or amphotericin B), antiviral agents (i.e., acyclovir), topical antiseptics (i.e., povidone-iodine), analgesics (i.e., lidocaine or benzocaine), or steroids (i.e., triamcinolone or hydrocortisone). Preferably, the second therapeutic agent is administered within 3 days, 1 day, 12 hours, 1 hour, or simultaneously with the trefoil peptide. The second therapeutic agent can be present in the same pharmaceutical composition as the trefoil peptide.

The invention also features pharmaceutical compositions suitable for delivering a trefoil peptide to the upper alimentary canal. Preferably, the pharmaceutical composition is an oral spray, an oral rinse (mouthwash), an ointment, a paste, a cream, a gel, chewing gum, a chewable tablet, a lozenge, or a bioerodable film. In one embodiment, the pharmaceutical compositions use bioerodable microspheres to encapsulate one or more of the therapeutic agents. In preferred embodiments of an oral spray, rinse, ointment, paste, gel, or bioerodable film, a mucoadhesive or viscosity-enhancing agent is present.

In preferred embodiments, the pharmaceutical composition further contains a second therapeutic. Preferred second therapeutic agents include anti-inflammatory agents, antibacterial agents (i.e., penicillins, cephalosporins, tetracyclines, or aminoglycosides), antifungal agents (i.e., nystatin or amphotericin B), antiviral agents (i.e., acyclovir), topical antiseptics (i.e., povidone-iodine), analgesics (i.e., lidocaine or benzocaine), or steroids (i.e., triamcinolone or hydrocortisone).

By “trefoil domain” is meant a polypeptide having a sequence substantially identical to any one of SEQ ID NOs:7-10, which correspond to the trefoil domains of hpS2₃₀₋₇₀, hSP1₃₀₋₇₁, hSP2₈₀₋₁₂₀, and hITF₂₄₋₆₄, respectively, and retain at biologic activity characteristic of trefoil peptides. The aligned polypeptide sequences of the four identified human trefoil domains are shown in FIG. 4. It is recognized in the art that one function of the six conserved cysteine residues is to impart the characteristic three-loop (trefoil) structure to the protein. The loop structure conforms to the general intrachain disulfide configuration of cys₁-cys₅ (corresponding to amino acid residues 25 and 51 of hITF; SEQ ID NO.:1), cys₂-cys₄ (corresponding to amino acid residues 35 and 50 of hITF; SEQ ID NO.:1), and cys₃-cys₆ (corresponding to amino acid residues 45 and 62 of hITF; SEQ ID NO.:1).

By “trefoil peptide (TP)” is meant any polypeptide having at least a trefoil domain (TD) and retaining a biological activity characteristic of trefoil peptides. Thus, preferred TPs may be any mammalian homolog or artificial polypeptide that are substantially identical to human spasmolytic polypeptide (hSP; also known as TFF2, GenBank Accession No. NM_{—}005423; SEQ ID NO.:5), human pS2 (also known as TFF1, GenBank Accession No. XM_{—}009779; SEQ ID NO.:3), human intestinal trefoil factor (hITF; also known as TFF3, SEQ ID NO.:1), and biologically active fragments of hSP, human pS2, and hITF. If desired, the TP may contain a cysteine residue outside of the trefoil domain suitable for disulfide bonding in the formation of homo- and heterodimers. Most preferably, the additional cysteine is C-terminal to the trefoil domain. Exemplary TPs include ITF₁₋₇₃, ITF₁₅₋₇₃, ITF₂₁₋₇₃, ITF₁₅₋₇₂, ITF₂₁₋₇₂, ITF₁₋₆₂, ITF₁₋₇₀, ITF₁₋₇₂, and ITF₂₁₋₇₃. Preferably, a TP is encoded by a nucleic acid molecule that hybridizes under high stringency conditions to the coding sequence of hITF (SEQ ID NO.:2), hSP (SEQ ID NO.:6), or hpS2 (SEQ ID NO.:4). TPs amenable to methods of this invention may exist as monomers, dimers, or multimers. For example, TP monomers may form an interchain disulfide linkage to form a dimer.

Mammalian trefoil peptides were discovered in 1982. One of the mammalian trefoil peptides, human intestinal trefoil factor (hITF; TFF3), has been characterized extensively, and is described in U.S. Pat. Nos. 6,063,755, and 6,221,840, hereby incorporated by reference. The other two known trefoil peptides are spasmolytic polypeptide (SP; TFF2) and pS2 (TFF1). Intestinal trefoil peptides, described extensively in the literature (e.g., Sands et al., Ann. Rev. Physiol. 58: 253-273, 1996), are expressed in the gastrointestinal tract and have a three-loop structure formed by intrachain disulfide bonds between conserved cysteine residues. These peptides protect the intestinal tract from injury and can be used to treat intestinal tract disorders such as peptic ulcers and inflammatory bowel disease. Homologs of these human polypeptides have been found in a number of non-human animal species. All members of this protein family, both human and non-human, are referred to herein as trefoil peptides. Human ITF will be referred to most extensively in this application; however, the activity of human ITF is common to each of the mammalian trefoil peptides.

By “co-formulated” is meant any single pharmaceutical composition which contains two or more therapeutic or biologically active agents.

By “pharmaceutical preparation” or “pharmaceutical composition” is meant any composition which contains at least one therapeutically or biologically active agent and is suitable for administration to a patient. For the purposes of this invention, pharmaceutical compositions suitable for delivering a therapeutic to the upper alimentary canal include, but are not limited to solutions and suspensions delivered either as an oral spray or rinse, pastes, gels, chewable tablets, sublingual, gingival, or buccal wafers and films, chewing gum, lozenges, and other compositions designed to be retained in the mouth for an extended period of time. Any of these formulations can be prepared by well known and accepted methods of art. See, for example, Remingtion: The Science and Practice of Pharmacy, 19^{th} edition, (ed. A R Gennaro), Mack Publishing Co., Easton, Pa., 1995.

By “microsphere” is meant a bioerodable polymeric pharmaceutical delivery device having a diameter of 5-100 μm and a hollow central core suitable for encapsulation of the therapeutic agent. Typically, the therapeutic agent is encapsulated at the time of microsphere formulation.

By “therapeutically effective amount” is meant an amount sufficient to provide medical benefit. When administering trefoil peptides to a human patient according to the methods described herein, a therapeutically effective amount is usually about 0.1-1000 mg of trefoil peptide per day. Preferably, the patient receives, 10 mg, 100 mg, 250 mg, or 750 mg of trefoil peptide each day. The total daily does can be divided into multiple individual doses.

By “upper alimentary canal” is meant the portion of the digestive system proximal to the cardiac sphincter (cardioesophageal sphincter) of the stomach. Specifically, the upper alimentary canal is meant to include the oral cavity and associated structures (e.g., the tongue, gingival and sublingual tissues, and the hard and soft palates) and the esophagus.

By “biologically active,” when referring to a TP is meant any polypeptide that exhibits an activity common to naturally occurring trefoil peptides. An example of a biological activity common to the family of trefoil peptides is the ability to alter gastrointestinal motility in a mammal. Other biological activities include mucopolysaccaride binding, maintenance of the mucosa, and repair of mucosal integrity upon injury (see, for example, Taupin et al., Proc. Natl. Acad. Sci, USA, 97:799-804, 1999).

By “high stringency conditions” is meant any set of conditions that are characterized by high temperature and low ionic strength and allow hybridization comparable with those resulting from the use of a DNA probe of at least 40 nucleotides in length, in a buffer containing 0.5 M NaHPO4, pH 7.2, 7% SDS, 1 mM EDTA, and 1% BSA (Fraction V), at a temperature of 65° C., or a buffer containing 48% fornamide, 4.8×SSC, 0.2 M Tris-Cl, pH 7.6, 1× Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well known by those skilled in the art of molecular biology. See, e.g., F. Ausubel et al., in Current Protocols in Molecular Biology, John Wiley & Sons, New York, N.Y., 1998, hereby incorporated by reference. Other features and advantages of the invention will be apparent from the following detailed description, and from the claims.

By “isolated DNA” is meant DNA that is free of the genes which, in the naturally-occurring genome of the organism from which the given DNA is derived, flank the DNA. Thus, the term “isolated DNA” encompasses, for example, cDNA, cloned genomic DNA, and synthetic DNA.

By “treating” is meant administering a pharmaceutical composition for prophylactic and/or therapeutic purposes. The active ingredients of the pharmaceutical composition can treat the primary indication (i.e., epithelial lesion) or secondary symptoms (e.g., concomitant infection, pain, or inflammation).

By “analgesic” is meant an agent which relieves pain by elevating the pain threshold without significantly disturbing the consciousness of the patient.

By “antimicrobial agent” is meant any compound that alters the growth of bacteria or fungi cells, or viruses whereby growth is prevented, stabilized, or inhibited, or wherein the microbes are killed. In other words, the antimicrobial agents can be microbiocidal or microbiostatic.

By “thermal burn” is meant injury to or destruction of at least the epithelial cell layer caused by exposure to excessive temperature. Thermal burns of the upper alimentary canal are usually caused by ingestion of overly-heated foods and liquids, or inhalation of super-heated air. Thermal burns are meant to include, but are not limited to, burns classified as first degree, second degree, and third degree burns.

By “chemical burn” is meant injury to or destruction of at least the epithelial cell layer caused by exposure to noxious chemicals. Typically, chemical exposures of the upper alimentary canal are caused by inhalation or ingestion.

By “antineoplastic therapy” is meant any treatment regimen used to treat cancer. Typical antineoplastic therapies include chemotherapy and radiation therapy.

### BRIEF DESCRIPTION OF DRAWINGS

FIGS. 1A-B show the amino acid sequence (Accession No. BAA95531; SEQ ID NO.:1) and cDNA sequence (GenBank Accession No. NM_{—}003226; SEQ ID NO.:2) of human intestinal trefoil factor, respectively.

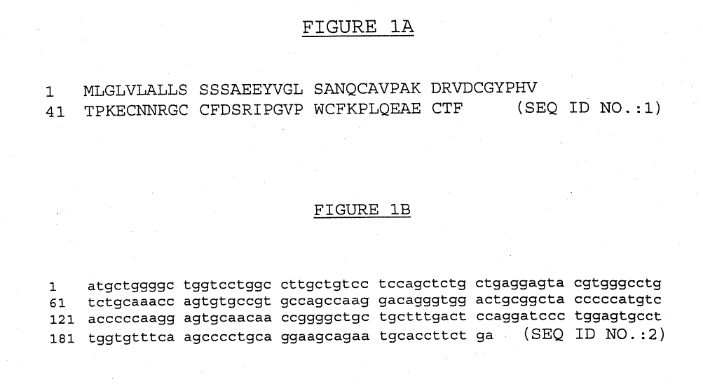

FIGS. 2A and 2B show the amino acid sequence (Accession No. NP_{—}0032166; SEQ ID NO.:3) and cDNA sequence (SEQ ID NO.:4) of human pS2 protein, respectively.

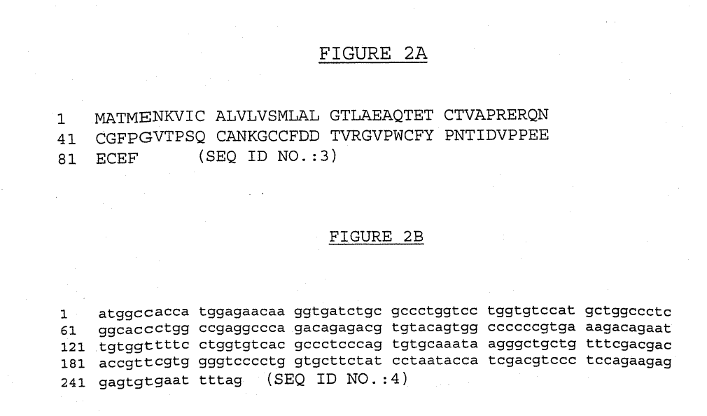

FIGS. 3A and 3B show the amino acid sequence (Accession No. 1909187A; SEQ ID NO.:5) and cDNA sequence (SEQ ID NO.: 6) of human spasmolytic polypeptide (SP).

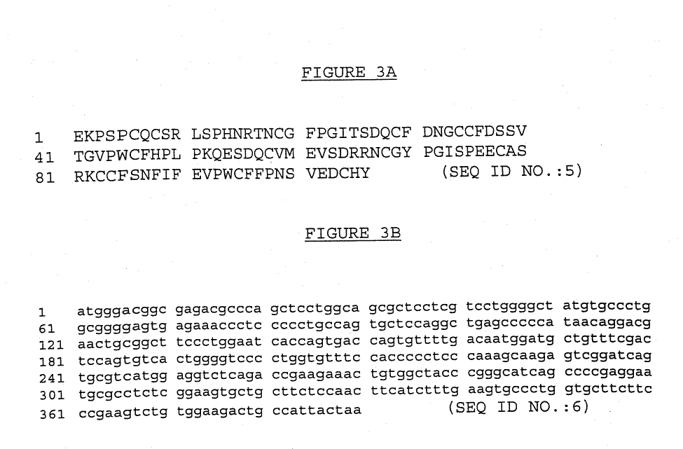

FIG. 4 is a multisequence alignment of trefoil domains (SEQ ID NOS.:7-10)/TFF1, SP/TFF2, and ITF/TFF3. X denotes any amino acid residue.

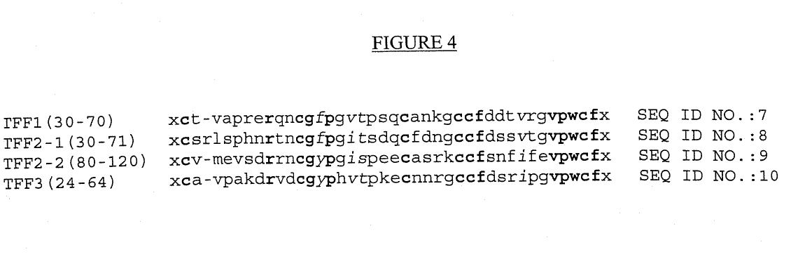

### DETAILED DESCRIPTION

The invention provides methods and compositions useful for the treatment of a wide range of lesions of the upper alimentary canal. The trefoil peptide therapy of this invention is particularly useful for treating epithelial lesions of the oral and esophageal mucosa, tongue, and gingival tissue.

We have discovered that epithelial lesions of the upper alimentary canal including the oral and esophageal mucosa, tongue, and gingival tissue can be treated by local administration of trefoil peptides. Thus, trefoil peptide therapy, according to the methods of this invention, can be delivered in any pharmaceutical composition which is useful for delivering therapeutics to the upper alimentary canal.

Pharmaceutical Preparations

Oral Sprays, Rinses, and Emulsions

Spray systems are particularly useful for delivering therapeutics to the upper alimentary canal. Suitable spray delivery systems include both pressurized and non-pressurized (pump actuated) delivery devices. The trefoil peptide-containing solution, delivered as an oral spray, is preferably an aqueous solution; however, organic and inorganic components, emulsifiers, excipients, and agents that enhance the organoleptic properties (i.e., flavoring agents or odorants) may be included. Optionally, the solution may contain a preservative that prevents microbial growth (i.e., methyl paraben). Although water itself may make up the entire carrier, typical liquid spray formulations contain a co-solvent, for example, propylene glycol, corn syrup, glycerin, sorbitol solution and the like, to assist solubilization and incorporation of water-insoluble ingredients. In general, therefore, the compositions of this invention preferably contain from about 1-95% v/v and, most preferably, about 5-50% v/v, of the co-solvent. When prepared as an spray, patients typically self-administer 1-5 times per day. The spray delivery system is normally designed to deliver 50-100 μl per actuation, and therapy may require 1-5 actuations per dose. The rheological properties of the spray formulation are optimized to allow shear and atomization for droplet formation. Additionally, the spray delivery device is designed to create a droplet size which promotes retention on mucosal surfaces of the upper alimentary canal and minimize respiratory exposure.

Compositions suitable for oral sprays can also be formulated as an oral rinse or mouthwash. Administration of trefoil peptides using these formulations is typically done by swishing, gargling, or rinsing the oral cavity with the formulation. Optionally, these formulations can be swallowed, providing trefoil peptide therapy to the esophagus, stomach, and/or intestines. This delivery method is particularly useful for treating patients suffering related disorders of the intestinal epithelium. For example, patients receiving antineoplastic chemotherapy, in addition to oral mucositis, frequently develop more distal lesions of the gastrointestinal tract such as lesions of the gastric and intestinal epithelium. It is well known that trefoil peptides, particularly ITF, are stable at stomach pH. Thus, swallowing a trefoil peptide-containing solution designed primarily for treating oral mucositis may also benefit lesions of the lower alimentary canal (i.e., stomach and intestines).

In an alternative formulation, the trefoil peptides and/or other therapeutics can be encapsulated in bioerodable microspheres rather than being dissolved in the aqueous phase of the formulation. A wide variety of microencapsulation drug delivery systems have been developed and many share similar polymeric compositions as used for bioerodable films (described below). Polymers commonly used in the formation of microspheres include, for example, poly-ε-caprolactone, poly(ε-caprolactone-Co-DL-lactic acid), poly(DL-lactic acid), poly(DL-lactic acid-Co-glycolic acid) and poly(ε-caprolactone-Co-glycolic acid) (see, for example, Pitt et al., J. Pharm. Sci., 68:1534, 1979).

Microspheres can be made by procedures well known in the art including spray drying, coacervation, and emulsification (see for example Davis et al. Microsphere and Drug Therapy, Elsevier, 1984; Benoit et al. Biodegradable Microspheres: Advances in Production Technologies, Chapter 3, Ed. Benita, S, Dekker, New York, 1996; Microencapsulation and Related Drug Processes, Ed. Deasy, Dekker, 1984, New York; U.S. Pat. No. 6,365,187). Preferably, the microspheres are bioadhesive or are prepared in formulations containing a bioadhesive excipient.

Other technical features of the trefoil peptide-containing solutions are easily modified to suit the specific pharmaceutical formulation and the clinical indication being treated. For example, the pH and osmolality of the formulation may be adjusted to confer trefoil peptide stability, while minimizing oral irritancy and sensitivity.

Ointments, Pastes, and Gels

Lesions of the oral and esophageal epithelium caused by trauma are amenable to trefoil peptide therapy delivered as an ointment, paste, or gel. The viscous nature of these types of preparations allows for direct application into the wound site. Optionally, the wound site can be covered with a dressing to retain the trefoil peptide-containing composition, protect the lesion from trauma, and/or absorb exudate. As discussed further below, these preparations are particularly useful to restore epithelial integrity following traumatic surgical procedures such as, for example, tooth extraction, tissue biopsy, or a tumor resection. Such viscous formulations may also have a local barrier effect thereby reducing irritation and pain.

Mucoadhesives

A mucoadhesive excipient can be added to any of the previously described pharmaceutical compositions. The mucoadhesive formulations coat the upper alimentary canal providing protection, inhibiting irritation, and accelerating healing of inflamed or damaged tissue. Mucoadhesive formulations also promote prolonged contact of the trefoil peptide with the mucosal epithelium. Mucoadhesive formulations suitable for use in pharmaceutical preparations delivered by mouth are well known in the art (e.g., U.S. Pat. No. 5,458,879). Particularly useful mucoadhesives are hydrogels composed of about 0.05-20% of a water-soluble polymer such as, for example, poly(ethylene oxide), poly(ethylene glycol), poly(vinyl alcohol), poly(vinyl pyrrolidine), poly(acrylic acid), poly(hydroxy ethyl methacrylate), hydroxyethyl ethyl cellulose, hydroxy ethyl cellulose, chitosan, and mixtures thereof. These polymeric formulations can also contain a dispersant such as sodium carboxymethyl cellulose (0.5-5.0%).

Other preferred mucoadhesive excipients for liquid compositions are ones that allow the composition to be administered as a flowable liquid but will cause the composition to gel in the upper alimentary canal, thereby providing a bioadhesive effect which acts to hold the therapeutic agents at the lesion site for an extended period of time. The anionic polysaccharides pectin and gellan are examples of materials which when formulated into a suitable composition will gel in the upper alimentary canal, owing to the presence of cations in the mucosal and salivary fluids. The liquid compositions containing pectin or gellan will typically consist of 0.01-20% w/v of the pectin or gellan in water or an aqueous buffer system.

Other useful compositions which promote mucoadhesion and prolonged therapeutic retention in the upper alimentary canal are colloidal dispersions containing 2-50% colloidal particles such as silica or titanium dioxide. Such formulations form as a flowable liquid with low viscosity suitable as a mouthwash or for generating a fine mist. However, the particles interact with glycoprotein, especially mucin, transforming the liquid into a viscous gel, providing effective mucoadhesion (e.g., U.S. Pat. Nos. 5,993,846 and 6,319,513).

Bioerodable Film Delivery Devices

The most simple bioerodable devices contain the therapeutic agent(s) incorporated into a solid, usually lipid-containing, film or tablet. The device is formulated to remain solid at room temperature, but melt at body temperature, releasing the incorporated therapeutics. Suitable formulations of this type include, for example, cocoa butter.

Polymeric film devices provide several advantages for therapeutic delivery to the oral cavity. Unlike rinses, pastes, gels, and other flowable compositions, a film device can reside for prolonged periods of time (i.e., hours to days) in the oral cavity and provide sustained release throughout its residency. Typically, the film is partially or completely bioerodable and contains a mucoadhesive layer to fasten the film to the oral mucosa. Film devices, in addition to its use for delivering therapeutics, can also provide protection against mechanical injury or microbial infection of a lesion site. This physical barrier function is particularly advantageous when treating conditions such as mucositis or aphthous stomatitis. Additionally, as discussed further below, a film device can be used to release trefoil peptide therapy directly onto the underlying mucosa, into the lumen of the oral cavity, or a combination of both.

Film devices consist of at least two layers; a mucoadhesive layer suitable for attaching the film to the oral mucosa and a bulk layer which contains the active therapeutic(s). Many suitable mucoadhesives are known in the art and are discussed above. Optionally, one or more therapeutics can also be provided in the adhesive layer.

The bulk layer of the composite delivery device may be made of one or more bioerodable polymeric materials. Suitable polymers include, for example, starch, gelatin, polyethylene glycol, polypropylene glycol, polyethylene oxide, copolymers of ethylene oxide and propylene oxide, copolymers of polyethylene glycol and polypropylene glycol, polytetramethylene glycol, polyether urethane, hydroxyethyl cellulose, ethyl cellulose, hydroxypropyl cellulose, hydroxypropylmethyl cellulose, alginate, collagen, polylactide, poly(lactide-co-glycolide) (PLGA), calcium polycarbophil, polyethymethacrylate, cellulose acetate, propylene glycol, polyacrylic acid, crosslinked polyacrylic acid, hydroxyethyl methacrylate/methyl methacrylate copolymer, silicon/ethyl cellulose/polyethylene glycol, urethane polyacrylate, polystyrene, polysulfone, polycarbonate, polyorthoesters, polyanhydrides, poly(amino acids), partially and completely hydrolyzed alkylene-vinyl acetate copolymers, polyvinyl chloride, polymers of polyvinyl acetate, polyvinyl alkyl ethers, styrene acrylonitrile copolymers, poly(ethylene terphthalate), polyalkylenes, poly(vinyl imidazole), polyesters and combinations of two or more of these polymers.

A particularly useful bulk layer polymer consists of PLGA and ethyl cellulose. PLGA is bioerodable and can be formulated to degrade over a wide range of conditions and rates. Ethyl cellulose is a water-insoluble polymer that can act as a plasticizer for the PLGA when a film is formed, but will be eroded in a bodily fluid. Due to its water-insolubility, it also has an effect on the degree and rate of swelling of the resultant film.

An optional third layer which is impermeable to the trefoil peptide can also be added to the wafer. Preferably, this barrier layer is also bioerodable. Suitable barrier layer polymers include ethyl cellulose, poly(acrylic acid), or other polyelectrolytes. In one configuration, the barrier layer is placed on the opposite side of the bulk layer relative to the adhesive layer, thereby directing the released therapeutic agent onto the contacted epithelium rather than being diluted in the lumenal fluid. This configuration is particularly useful for treating discrete lesions (i.e., mucositis or aphthous stomatitis) of the tongue, sublingual tissue, or buccal mucosa. In an alternative configuration of the film device, the barrier layer is placed between the bulk layer and the adhesive layer. This configuration directs therapeutic release into the lumen of the oral cavity and is useful for treating more diffuse lesions of the tongue, oral cavity, and esophagus. The configuration is also useful for delivering therapeutics which are cytotoxic when administered at high concentrations because it has the effect of shielding the underlying tissue from direct contact with the therapeutic-containing film.

Chewable Tablets, Lozenges, and Confectionaries

Preparing a trefoil peptide-containing composition as a chewable tablet, lozenge, or a confectionary such as chewing gum provides several advantages to traditional drug delivery vehicles. First, prolonged contact and sustained release at the target site (mouth and esophagus) is achieved. Second, such formulations often results in higher patient compliance, especially when administering trefoil peptides to children.

Formulations for chewable tablets are well known and typically contain a base of sugar, starch, or lipid and a flavoring agent. An exemplary formulation for a chewable tablet is provided below.

Chewable ITF Tablet Formulation (Per Tablet)

Intestinal trefoil factor—300 mg
Mannitol—675 mg
Microcrystalline cellulose—75 mg
Corn starch—30 mg
Calcium sterate—22 mg
Flavoring Agent (i.e., sodium saccharin or peppermint oil)

The incorporation of therapeutics into chewing gum and other confectionary style formulations is known in the art (e.g., U.S. Pat. No. 5,858,391).

Therapeutics Agents

Trefoil Peptides

The therapeutic trefoil peptide(s) are typically mammalian intestinal trefoil peptides. Preferably, human intestinal trefoil peptides are used; however, trefoil peptides from other species including rat, mouse, and non-human primate, may be used. Typically, the trefoil peptide is intestinal trefoil factor (ITF); however, spasmolytic polypeptide (SP), or pS2 are also useful. Particularly useful ITF fragments that retain biological activity include the polypeptide corresponding to amino acid residues 15-73 of SEQ ID NO:1 (ITF₁₅₋₇₃) and amino acid residues 21-73 of SEQ ID NO:1 (ITF₂₁₋₇₃). Other useful ITF fragments are formed following cleavage of the C-terminal phenylalanine residue (i.e., ITF₁₋₇₂, ITF₁₅₋₇₂, and ITF₂₁₋₇₂).

The trefoil peptides are present in the compositions of the invention at a concentration of between 0.1-1000 mg/ml, depending on the nature and condition of the lesion being treated, the anticipated frequency and duration of therapy, and the type of pharmaceutical composition used to deliver the trefoil peptide. Typically, therapy is designed to deliver 0.1-500 mg of trefoil peptide per day to the patient.

Anti-Inflammatory Agents

Any suitable anti-inflammatory agent can be formulated in the compositions of the invention, at concentrations known for these agents. Many of the most useful anti-inflammatory agents also have analgesic and/or antipyretic properties. Anti-inflammatory agents suitable for co-formulation with a trefoil peptide include, for example, acetaminophen, aspirin (acetylsalicylic acid), ibuprofen, phenylbutazone, indomethacin, sulindac, diclofenac, and naproxen.

Antimicrobial Agents

Any of the many known microbial agents can be used in the compositions of the invention at concentrations generally used for these agents. Antimicrobial agents include antibacterials, antifungals, antivirals, and other topical antiseptics.

Examples of antibacterial agents (antibiotics) include the penicillins (e.g., penicillin G, ampicillin, methicillin, oxacillin, and amoxicillin), the cephalosporins (e.g., cefadroxil, ceforanid, cefotaxime, and ceftriaxone), the tetracyclines (e.g., doxycycline, minocycline, and tetracycline), the aminoglycosides (e.g., amikacin, gentamycin, kanamycin, neomycin, streptomycin, and tobramycin), the macrolides (e.g., azithromycin, clarithromycin, and erythromycin), the fluoroquinolones (e.g., ciprofloxacin, lomefloxacin, and norfloxacin), and other antibiotics including chloramphenicol, clindamycin, cycloserine, isoniazid, rifampin, and vancomycin.

Antiviral agents are substances capable of destroying or suppressing the replication of viruses. Examples of anti-viral agents include 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon, adenine arabinoside, protease inhibitors, thymadine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir.

Antifungal agents include both fungicidal and fungistatic agents such as, for example, amphotericin B, butylparaben, clindamycin, econaxole, fluconazole, flucytosine, griseofulvin, nystatin, and ketoconazole.

Topical antiseptics include agents such as, for example, povidone-iodine and benzalkonium chloride.

Analgesics and Anesthetics

Any of the commonly used topical analgesics can be used in the compositions of the invention. The analgesic is present in an amount such that there is provided to the oral lesion a topical concentration of between one-half and five percent concentration for lidocaine (5-50 mg/ml in 20-40 ml per dose of liquid). Examples of other useful anesthetics include procaine, lidocaine, tetracaine, dibucaine, benzocaine, p-buthylaminobenzoic acid 2-(diethylamino) ethyl ester HCl, mepivacaine, piperocaine, and dyclonine.

Other analgesics include opioids such as, for example, morphine, codeine, hydrocodone, and oxycodone. Any of these analgesics may also be co-formulated with other compounds having analgesic or anti-inflammatory properties, such as acetaminophen, aspirin, and ibuprofen.

Steroids

Steroids are commonly used to treat lesions of the upper alimentary canal. For example, oral aphthous stomatitis is typically treated using a paste preparation of triamcinolone (0.1%), hydrocortisone, fluticasone, or beclomethasone.

Conditions of the Upper Alimentary Canal Treated Using Trefoil Peptides

Mucositis

Mucositis is a common condition of the oral cavity which is characterized by inflammation of the mucous membranes. The condition is frequently caused by antineoplastic therapy, including chemotherapy and local radiation therapy. Symptoms of mucositis include ulcerations, redness, and swelling, and is associated with epithelial cell injury and death. Patients suffering from severe mucositis are susceptible to dehydration and malnutrition because mucositis pain limits dietary intake. In severe cases, mucositis can be so debilitating that patients may require prolonged hospitalization, parenteral nutrition, and narcotic pain medication. Additionally, destruction of the mucosal epithelium increases a patient's susceptibility to local and systemic infection. Disruption of the barrier function permits entry of microorganisms and microbial products normally retained in the gut lumen. Thus, pharmaceutical preparations which reduce the adverse effects associated with chemotherapy will improve the patient's quality of life, compliance with self-medication, and may permit administration of higher chemotherapeutic doses. Typically, mucositis is treated using a trefoil peptide-containing rinse or oral spray which the patient self-administers 1-5 times per day. The aqueous solution preferably contains a mucoadhesive and an anti-inflammatory agent. Other therapeutics, such as an topical analgesic agent (e.g., lidocaine) may also be present. Alternatively, if the lesions are few in number and spatially localized, a trefoil peptide-containing film device an be placed directly over the lesions.

Tooth Extraction

Trefoil peptide-containing compositions of the invention are used to lessen complications and speed healing of the wound created by the extraction of a tooth. An oral rinse, paste, ointment, or gel, as described above, is applied to the site of extraction immediately following the procedure and then 1-4 times per day, as needed, until epithelial regrowth is complete. Preferably, a topical analgesic is included in the formulation to relieve the temporary discomfort cause by the trauma of extraction. As a prophylactic measure, antibiotic agents may also be included in the formulation.

Gingivitis

Gingivitis is most commonly a chronic disease requiring ongoing treatment, in some cases for months or even years. The trefoil peptide-containing compositions of the invention can be employed to treat gingivitis, alone or in conjunction with other treatments, particularly with an anti-microbial agent, and most commonly with an antibacterial agent. An oral trefoil peptide-containing rinse is swished in the patient's mouth at least once every 2-3 days, but as often as thrice daily, over a 3-4 week period, and the regimen is repeated as needed. Alternatively, the trefoil peptide is formulated into a gel or toothpaste. In severe cases, a viscous gel or ointment having a high trefoil peptide concentration is applied directly to the wound via a pledget with a stick applicator.

Trefoil peptide-containing compositions can also be delivered in biodegradable drug delivery systems capable of formation of films applied below the gum line (described in U.S. Pat. Nos. 5,945,115 and 5,990,194. A biodegradable polymer, admixed with the trefoil peptide, is provided where the polymer can be injected in as a free-flowing solution below the gum line using a syringe. The polymer solution then, in situ, forms a solid biodegradable implant.

Aphthous Stomatitis

At the first indication of an outbreak of aphthous stomatitis (generally, the first twinge of pain), the patient swishes the mouth with a trefoil peptide-containing rinse, 1-4 times per day until the ulcer heals (generally 5-10 days). A trefoil peptide-containing gel can also be applied to the ulcer, in the same manner that steroid-containing gels are currently used. In addition, a gel can contain both a trefoil protein and a steroid known to be effective for aphthous stomatitis treatment. A direct application of more concentrated material can be directly applied to the wound via a pledget with a stick applicator. Alternatively, the lesion can be treated directly by applying a bioerodable film device containing both a trefoil peptide and a steroid (i.e., triamcinolone) directly to the lesion. Any formulation useful for treating aphthous stomatitis can also, optionally, contain a local anesthetic (i.e., lidocaine or benzocaine).

Behcet's Disease

Behcet's Disease is a rare, multi-system rheumatic disorder characterized by systemic vasculitis. One of the most frequent symptoms of Behcet's Disease is recurrent oral ulcerations which resemble aphthous lesions. Currently, treatment for Behcet's Disease is palliative, not curative. Thus, the trefoil peptides can be used to treat lesions of the upper alimentary canal in conjunction with currently available Behcet's Disease therapies including, for example, interferon alpha 2A and 2B, levamisole, cyclosporine, cyclophosphamide, and colchicine.

Oral Biopsy and Oral Surgery

In cases in which an oral neoplasm is suspected or known to be malignant, a biopsy or a curative resection is performed using a needle or a scalpel, resulting in an open wound. The surgical area, susceptible to infection and inflammation, is treated by rinsing with a trefoil peptide-containing solution 1-4 times per day. Preferably, an analgesic, an anti-inflammatory, and an antibiotic are included in the formulation Alternatively, a more concentrated gel, paste, or ointment may be directly applied to the lesion site. For post-operative treatment following resection of a malignancy, a topically active chemotherapeutic can be including in the trefoil peptide-containing composition.

Thermal and Chemical Burns

Trauma to the upper alimentary canal is frequently caused by exposure to excessive heat or noxious chemicals. Thermal burns to the upper alimentary canal are frequently mild in nature (i.e., first or second degree burns), resulting from the ingestion of overheated food or drink. More severe thermal burns of the oral mucosa and upper esophagus can be caused by inhalation of super heated air and are frequently observed in firefighters or victims of house or forest fires.

Chemical exposure can also damage the mucosa of the upper alimentary canal. Mild mucosal irritations and burns are often caused by ingestion of acidic food (i.e., fruits). More severe chemical burns are usually associated with accidental industrial or occupational exposures.

The trefoil peptide-containing pharmaceutical formulations described herein are useful for treating thermal and chemical burns of the upper alimentary canal. Preferably, viscous liquid or gel formulation containing a mucoadhesive is used to prolong mucosal exposure to the trefoil peptide. Alternatively, a sustained release formulation, such as a bioerodable film, is used. Topical analgesics and antimicrobial agents are the most preferred secondary therapeutics to be co-administered.

Production of Trefoil Peptides

Trefoil peptides can be produced by any method known in the art for expression of recombinant proteins. Nucleic acids that encode trefoil peptides may be introduced into various cell types or cell-free systems for expression thereby allowing large-scale production, purification, and patient therapy.

Eukaryotic and prokaryotic trefoil peptide expression systems may be generated in which a trefoil peptide gene sequence is introduced into a plasmid or other vector, which is then used to transform living cells. Constructs in which the trefoil peptide cDNA contains the entire open reading frame inserted in the correct orientation into an expression plasmid may be used for protein expression. Prokaryotic and eukaryotic expression systems allow for the expression and recovery of trefoil peptide fusion proteins in which the trefoil peptide is covalently linked to a tag molecule which facilitates identification and/or purification. An enzymatic or chemical cleavage site can be engineered between the trefoil peptide and the tag molecule so that the tag can be removed following purification.

Typical expression vectors contain promoters that direct the synthesis of large amounts of mRNA corresponding to the inserted trefoil peptide nucleic acid in the plasmid-bearing cells. They may also include a eukaryotic or prokaryotic origin of replication sequence allowing for their autonomous replication within the host organism, sequences that encode genetic traits that allow vector-containing cells to be selected for in the presence of otherwise toxic drugs, and sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

Expression of foreign sequences in bacteria, such as Escherichia coli, requires the insertion of a trefoil peptide nucleic acid sequence into a bacterial expression vector. Such plasmid vectors contain several elements required for the propagation of the plasmid in bacteria, and for expression of the DNA inserted into the plasmid. Propagation of only plasmid-bearing bacteria is achieved by introducing, into the plasmid, selectable marker-encoding sequences that allow plasmid-bearing bacteria to grow in the presence of otherwise toxic drugs. The plasmid also contains a transcriptional promoter capable of producing large amounts of mRNA from the cloned gene. Such promoters may be (but are not necessarily) inducible promoters that initiate transcription upon induction. The plasmid also preferably contains a polylinker to simplify insertion of the gene in the correct orientation within the vector. Biologically active trefoil peptides also can be produced using a Pichia yeast expression system (see, for example, U.S. Pat. Nos. 4,882,279 and 5,122,465; hereby incorporated by reference).

Mammalian cells can also be used to express a trefoil peptide. Stable or transient cell line clones can be made using trefoil peptide expression vectors to produce the trefoil peptides in a soluble (truncated and tagged) form. Appropriate cell lines include, for example, COS, HEK293T, CHO, or NIH cell lines.

Once the appropriate expression vectors are constructed, they are introduced into an appropriate host cell by transformation techniques, such as, but not limited to, calcium phosphate transfection, DEAE-dextran transfection, electroporation, microinjection, protoplast fusion, or liposome-mediated transfection. The host cells that are transfected with the vectors of this invention may include (but are not limited to) E. coli or other bacteria, yeast, fungi, insect cells (using, for example, baculoviral vectors for expression in SF9 insect cells), or cells derived from mice, humans, or other animals. In vitro expression of trefoil peptides, fusions, or polypeptide fragments encoded by cloned DNA may also be used. Those skilled in the art of molecular biology will understand that a wide variety of expression systems and purification systems may be used to produce recombinant trefoil peptides and fragments thereof. Some of these systems are described, for example, in Ausubel et al. (Current Protocols in Molecular Biology, John Wiley & Sons, New York, N.Y. 2000, hereby incorporated by reference).

Transgenic plants, plant cells and algae are also particularly useful for generating recombinant trefoil peptides for use in the methods and compositions of the invention. For example, transgenic tobacco plants or cultured transgenic tobacco plant cells expressing a trefoil peptide can be created using techniques known in the art (see, for example, U.S. Pat. Nos. 5,202,422 and 6,140,075). Transgenic algae expression systems can also be used to produce recombinant trefoil peptides (see, for example, Chen et al., Curr. Genet. 39:365-370, 2001).

Once a recombinant protein is expressed, it can be isolated from cell lysates using protein purification techniques such as affinity chromatography. Once isolated, the recombinant protein can, if desired, be purified further by e.g., high performance liquid chromatography (HPLC; e.g., see Fisher, Laboratory Techniques In Biochemistry And Molecular Biology, Work and Burdon, Eds., Elsevier, 1980).

Polypeptides of the invention, particularly short trefoil peptide fragments can also be produced by chemical synthesis using, for example, Merrifield solid phase synthesis, solution phase synthesis, or a combination of both (see, for example, the methods described in Solid Phase Peptide Synthesis, 2nd ed., 1984, The Pierce Chemical Co., Rockford, Ill.). Optionally, peptide fragments are then be condensed by standard peptide assembly chemistry.

### EXAMPLE 1

### Mucositis Treatment for Patients Receiving Antineoplastic Therapy

Trefoil peptide therapy is initiated prior to antineoplastic therapy (i.e., chemotherapy or radiation therapy), as a prophylactic to delay or prevent the onset of mucositis. Preferably, the patient begins trefoil peptide therapy three days prior to the first dose of antineoplastic therapy. During the prophylactic stage, the patient rinses the oral cavity with a trefoil peptide-containing solution. Alternatively, for convenience, the trefoil peptide is provided as a concentrated oral spray. Preferably, the patient swallows the solution, providing protection for the epithelial cells of the esophagus and lower gastrointestinal tract. Rinsing with and swallowing the trefoil peptide-containing solution continues at least twice daily until oral or esophageal mucositis is detected.

In patients with existing mucositis, epithelial healing is promoted using trefoil peptide therapy as described above. Palliative therapy is provided using benzocaine (a local anesthetic), and nystatin (an antifuingal). The trefoil peptide can be co-formulated with the benzocaine and nystatin. For example, the patient swishes an oral rinse solution (mouthwash), containing all therapeutic agents, 1-5 times each day. Alternatively, the trefoil peptide can be provided in a concentrated oral spray, with or without benzocaine and the nystatin is administered in an oral rinse.

The oral rinse solutions can either be swallowed or spit out. If swallowed, an antacid may also be included in the formulation. Other useful therapeutics which provide palliative therapy include anti-inflammatories (e.g., ibuprofen) and other anti-microbial agents. Exemplary oral rinses useful for treating chemotherapy-induced mucositis are provided below, but are not intended to be limiting. A skilled physician or pharmacist will immediately recognize appropriate substitutions, additions, and deletions that can be made to these formulations.

Rinse#1: Mix equal parts of:
(i) diphenhydramine elixir (Benadryl®)
(ii) kaolin-pectin suspension (Kaopectate®)
(iii) viscous lidocaine HCl (2%)
(iv) nystatin (oral suspension; 100,000 iu/ml)
(v) ITF (2.5 mg/ml)
preferably swallowed after swishing

Rinse#2: Mix equal parts of:
(i) diphenhydramine elixir (Benadryl®)
(ii) Maalox® (MgOH & AIOH; 40 mg/ml)
(iii) viscous lidocaine HCl (2%)
(iv) ITF (2.5 mg/ml)
preferably swallowed after swishing

### Other Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A composition comprising an intestinal trefoil peptide and a mucoadhesive excipient.

2. The composition of claim 1, wherein said mucoadhesive comprises a water-soluble polymer.

3. The composition of claim 1, wherein said mucoadhesive comprises a polysaccharide.

4. The composition of claim 1, wherein said composition comprises a solution.

5. The composition of claim 1, wherein said composition comprises a suspension.

6. The composition of claim 1, wherein said intestinal trefoil peptide is spasmolytic polypeptide, pS2, or intestinal trefoil factor.

7. The composition of claim 6, wherein said intestinal trefoil peptide is intestinal trefoil factor.

8. The composition of claim 7, wherein said intestinal trefoil factor is human intestinal trefoil factor.

9. The composition of claim 1, wherein said composition further comprises a second therapeutic agent.

10. The composition of claim 9, wherein said second therapeutic agent is an anti-inflammatory agent, an antibacterial agent, an antiviral agent, an antifungal agent, a topical antiseptic, an analgesic, or a steroid.

11. The composition of claim 10, wherein said anti-inflammatory agent is acetaminophen, aspirin, ibuprofen, phenylbutazone, indomethacin, sulindac, diclofenac, or naproxen.

12. The composition of claim 10, wherein said antibacterial agent is a penicillin, a cephalosporin, a tetracycline, an aminoglycoside, a macrolide, a fluoroquinolone, chloramphenicol, clindamycin, cycloserine, isoniazid, rifampin, or vancomycin.

13. The composition of claim 10, wherein said antiviral agent is 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon, adenine arabinoside, a protease inhibitor, a thymadine kinase inhibitor, a sugar or glycoprotein synthesis inhibitor, a structural protein synthesis inhibitor, an attachment or adsorption inhibitor, acyclovir, penciclovir, valacyclovir, or ganciclovir.

14. The composition of claim 10, wherein said antifungal agent is amphotericin B, butylparaben, clindamycin, econaxole, fluconazole, flucytosine, griseofuilvin, nystatin, or ketoconazole.

15. The composition of claim 10, wherein said topical antiseptic is povidone-iodine or benzalkonium chloride.

16. The composition of claim 10, wherein said analgesic is procaine, lidocaine, tetracaine, dibucaine, benzocaine, p-buthylaminobenzoic acid 2-(diethylamino) ethyl ester HCl, mepivacaine, piperocaine, dyclonine, morphine, codeine, hydrocodone, or oxycodone.

17. The composition of claim 10, wherein said steroid is triamcinolone, hydrocortisone, fluticasone, or beclomethasone.

18. The composition of claim 1, wherein said intestinal trefoil peptide is present in a therapeutically effective amount.

19. The composition of claim 18, wherein said composition comprises 10 mg of intestinal trefoil peptide.

20. The composition of claim 18, wherein said composition comprises 100 mg of intestinal trefoil peptide.
